(19) United States
(12) Patent Application Publication
(10) Pub. No.: US 2007/0276310 A1
(43) Pub. Date: Nov. 29, 2007

(54) Therapeutic sleeve for applying compression to a body part

(76) Inventors: Moses A. Lipshaw, San Diego, CA (US); Sandra Anne Shaw, Coronado, CA (US)

Correspondence Address: GORDON & REES LLP, 101 WEST BROADWAY, SUITE 1600, SAN DIEGO, CA, 92101, US

(21) Appl. No.: 11/441,730

(22) Filed: May 26, 2006

(51) Int. Cl. A61F 13/06; A61F 13/00

(52) U.S. Cl. 602/62; 602/61

(57) ABSTRACT

A therapeutic sleeve for applying pressure to a body part, having: a tubular semi-elastic body; at least one seam extending along the tubular semi-elastic body, the at least one seam defining a region of narrowed thickness of the tubular semi-elastic body; and an access opening through a side of the tubular semi-elastic body. Optionally, the stitches may be disposed in stitched sections that are separated end to end from one another.

### TECHNICAL FIELD

The present invention relates to devices that apply therapeutic compression to body parts; for example, during edema and lymphedema treatments.

### BACKGROUND OF THE INVENTION

Edema is the result of an imbalance in the filtration system between the capillary and interstitial spaces. Lymphedema occurs when a protein-rich fluid accumulates in the interstitium as a result of lymphatic block or dysfunction. (A. Mallick and A. R. Bodenham, Disorders of the lymph circulation: their relevance to anesthesia and intensive care, British Journal of Anaesthesia, 2003, 91 (2): 265-72.) These are chronic conditions for most patients with swelling of limbs. Compression applied to a body part, such as a limb, is essential for resolving many circulatory disorders, including edema and lymphedema.

The typical treatment of lymphedema involves some form of compression to induce the movement of interstitial fluid and lymph out of the limb. During the treatment phase and often during the maintenance phase of lymphedema, the most common form of compression is applied through the use of short-stretch bandages. These bandages are applied over a material, such as a sleeve, which is first wrapped around the limb. The sleeve is necessary because it provides protection, distributes compression evenly, and adds comfort.

The application of compression to the limbs, either during the day, overnight, or both is necessary to relieve swelling. The typical treatment of lymphedema involves some form of compression to induce the movement of interstitial fluid and lymph out of the limb. Moreover, compression restores circulation, relieves swelling, treats pain, heals ulcers, and treats varicose veins. For most patients with swelling of limbs, these disorders are chronic.

During the treatment phase of lymphedema and often the maintenance phase, the most common form of compression is applied through the use of short-stretch bandages. These bandages are applied over a soft layer of foam, which is first wrapped around the limb. The semi elastic material is wrapped around the garment/limb by pulling at the same tension going around the limb in overlapping layers. Depending on the amount of compression needed and the therapist, the number of layers may vary. Wrapping the foam around the limb is time consuming and often difficult for a patient to do themselves. This foam is necessary though as it provides protection, distributes compression evenly, and adds comfort.

In certain areas of a limb, such as the ankle or the wrist, there may be overhangs of swollen tissue. To prevent soreness in these overlapping areas, therapists may often apply additional pieces of foam that target these locales. These foam pieces provide more comfort to the patient and/or provide compression to these specific places. Again these foam pieces need to be wrapped over to be held in place before bandaging and can not be added once the limb is covered.

Patients have observed that stockings, wraps, and bandaging systems made entirely of elastic materials are uncomfortable. Fully elastic devices deliver an unchanging level of pressure, which alternately feels either “too tight” or “too loose” to the patient depending on the patient's position. These elastic systems also do not resist small changes in limb circumference, and hence do not provide the fluctuating pressures that are needed to assist with pumping fluid out of the body part.

Elastic and inelastic garments have been employed in compression therapy of the limbs. (U.S. Pat. Nos. 4,084,584; 4,474,573; 5,385,036; 5,449,341; 5,830,237; and 6,807,683)

A possible method of providing compression to a limb is through the use of a substantially non-elastic compression garment, such as those manufactured by CircAid Medical Products of San Diego Calif., or devices such as described in U.S. Pat. No. 5,653,244. These products utilize a series of d-rings/straps or interlocking bands to apply compression. The garment is applied over the therapeutic sleeve and its bands/straps are simply tightened using the same tension on each. The amount of compression depends on the patient.

U.S. Pat. No. 5,976,099 describes an enclosed sleeve that is applied to the limb prior to compression, providing a base on which to apply compression. However, this device consists of a multiplicity of particles that fill the sleeve. This results in localized areas of high and low pressure on the limb when compression is applied, which is not always desired.

Therapeutic sleeves are therefore needed that are easy to apply, and that allow compression that is both sustained (in that significant long-term changes in limb volume can be accommodated), and dynamic (such that short-term changes to internal pressure can be countered). To this end, therapeutic sleeves are needed that provide the ability to apply and adjust compression as quickly and easily as possible. Therapeutic sleeves are also needed that are inelastic enough to allow compression levels that respond dynamically to changes in patients' compression requirements, while still being elastic enough so that the device does not readily loose appropriate compression. A need also exists for therapeutic sleeves that can be applied to parts of the body that have varying circumference and that are comfortable to wear throughout the day and in different postures.

Furthermore, a need exists for a therapeutic sleeve that permits additional support or compression to be applied at body joints such as wrists or ankles.

In addition, a need exists for a therapeutic sleeve that maintains its semi-elastic properties while contouring to the shape of a body limb. Preferably as well, such a therapeutic sleeve will maintain its elastic properties in both the longitudinal (length) and lateral (width) directions.

### SUMMARY OF THE INVENTION:

The present invention provides a therapeutic sleeve to be used underneath a compression device on a patient's arm or leg. (It is to be understood, however, that the present invention is not so limited, and can be applied to any body part.) The present therapeutic sleeve, which has inner and outer surfaces, may be made from a unitary, flexible, semi-elastic foam material. The tubular semi-elastic body is dimensioned to wrap around a body part having proximal and distal edges which are open (through which an arm or leg or other body part is received). The tubular semi-elastic body may optionally be made of foam, and may be covered by a shell (such as a cotton-Lycra shell). It may also optionally include a fabric laminate on the foam. In preferred aspects, the edges of the unitary, flexible, semi-elastic foam material may be sewn together to form the tubular sleeve shape. It is to be understood, however, that other systems including hook and eye fasteners, might be employed in place of stitching.

In one preferred aspect, the present invention provides a therapeutic sleeve for applying pressure to a body part, having: a tubular semi-elastic body; at least one seam extending along the tubular semi-elastic body (defining a region of narrowed thickness of the tubular semi-elastic body); and an access opening through a side of the tubular semi-elastic body.

The at least one seam is preferably formed by stitches passing through the tubular semi-elastic body. The stitches may extend longitudinally along the length of the tubular semi-elastic body. As will be explained, such seams define regions of narrowed thickness of the tubular semi-elastic body. The elastic nature of sleeve and the specific seam patterns that keep the sleeve elastic help the sleeve cling to the body part as limb volume decreases in diameter over time.

Preferably, the access opening is dimensioned to be disposed near a body joint (such as a wrist or ankle) when the therapeutic sleeve is received over the body part. Padding may be inserted directly through the access opening to provide additional support at the body joint.

The present invention may further comprise an openable slit extending longitudinally from an end of the tubular semi-elastic body. This openable slit may simply be a reinforced seam that can be cut open. As will be shown, the openable slit permits the sleeve to be dimensioned to fit body parts of different lengths. Thus, the present invention can easily be fitted for a particular patient. In one optional embodiment, the openable slit extends along the entire length of the sleeve such that the entire sleeve can be opened to wrap around the patient's limb and overlap upon itself. This may be useful in treating patients as their limbs decrease in diameter over time.

The present invention also provides a therapeutic sleeve for applying pressure to a body part, comprising: a tubular semi-elastic body; at least one seam defining a region of narrowed thickness extending along the tubular semi-elastic body, the seam being formed by stitches passing through the tubular semi-elastic body, wherein the stitches are disposed in stitched sections that are separated end to end from one another.

In various aspects, the stitched sections may be separated end to end from one another longitudinally along the length (or laterally across the width) of the tubular semi-elastic body. In various embodiments, the stitched sections may be disposed in a straight line, a zig-zag pattern, or at right angles to one another. It is to be understood that these stitching patterns are merely exemplary and that the present invention is not so limited. Rather, other possibilities of stitching patterns are also contemplated within the scope of the present invention. As will be shown, the present stitching patterns advantageously allow the sleeve to maintain its semi-elastic properties in perpendicular longitudinal and lateral directions.

The present invention also provides a method of using a therapeutic sleeve for applying pressure to a body part, by: receiving a body part within a tubular semi-elastic body having at least one seam extending therealong (defining a region of narrowed thickness of the tubular semi-elastic body); and then inserting padding through an access in a side of the tubular semi-elastic body.

The present invention also provides a method of sizing a therapeutic sleeve to a body part, by: receiving a body part within a tubular semi-elastic body having at least one seam extending therealong, the at least one seam defining a region of narrowed thickness of the tubular semi-elastic body; and then opening an end portion of the at least one seam. For example, opening the end portion of the seam may be performed by simply cutting open an end portion of a reinforced seam. In optional embodiments, the entire sleeve may be opened by cutting open a reinforced seam passing along the entire length of the tubular body.

An advantage of the present invention is that it provides a semi-elastic, unitary, therapeutic sleeve that can be easily applied to a body part without the need to fasten the therapeutic sleeve around the body part. The present therapeutic sleeve is a soft, tubular, foam, continuous semi-elastic sleeve to be applied prior to compression in order to provide a simpler, more cost effective way of applying compression. The present therapeutic sleeve provides a smooth, conforming fit by incorporating semi-elastic foam materials, and is thus comfortable to wear, and does not slip on the limb.

Advantageously as well, the present therapeutic sleeve can be manufactured with reduced labor and material costs, as compared to more complex designs. In addition, the present therapeutic sleeve can be fabricated for short or long-term use depending on the materials used.

Other features and advantages of the present invention will become more fully apparent from the following detailed description of preferred embodiments, the appended claims, and the accompanying drawings in which:

### BRIEF DESCRIPTION OF THE DRAWINGS

FIG. 1 is a plan view of the outer surface of an arm version of the present therapeutic sleeve showing the shape of the material before it is assembled into a tubular sleeve, showing stitches therealong.

FIG. 2 is a perspective view of the assembled arm version of the present therapeutic sleeve showing an access opening near the wrist.

FIG. 3 is a perspective view of the arm version of the present therapeutic sleeve showing the positioning of the device on a patient's arm.

FIG. 4 is a sectional view of the present therapeutic sleeve taken along line 4-4 in FIG. 3, showing the advantages of the seams when the sleeve is received over a patient's arm.

FIG. 5 is a plan view of the outer surface of a leg version of the present therapeutic sleeve showing the shape of the material before it is assembled into a tubular sleeve, showing stitches passing therealong, a cut-away knee region, and an optional tongue. This illustrated embodiment extends from above the patient's knee to the patient's foot.

FIG. 6 is a perspective view of a leg version of the present therapeutic sleeve showing an access opening near the ankle. This illustrated embodiment extends from below the patient's knee to the patient's foot.

FIG. 7 is perspective view of the leg version of the present therapeutic sleeve showing the positioning of the device on the leg, and showing the functioning of the optional tongue.

FIG. 8A illustrates an operator cutting open a reinforced seam to size an arm version of the therapeutic sleeve.

FIG. 8B is a perspective view of the of the resulting sized therapeutic sleeve of FIG. 8A.

FIG. 8C is a view similar to FIG. 8B, but with the seam opened down to near the user's hand.

FIGS. 9A to 9D show close-up views of the stitching pattern on the therapeutic sleeves, as follows:

FIG. 9A is a plan view of a zig-zag stitching pattern on a portion of the therapeutic sleeve;

FIG. 9B is a plan view of a straight line stitching pattern on a portion of the therapeutic sleeve;

FIG. 9C is a plan view of an alternate stitching pattern on a portion of the therapeutic sleeve; and

FIG. 9D is a plan view of a right angled stitching pattern on a portion of the therapeutic sleeve.

FIG. 10 is a perspective view of an arm embodiment of the present therapeutic sleeve cut fully open along a reinforced seam.

FIG. 11 is a sectional elevation view through a patient's leg, showing a therapeutic sleeve having greater thickness in the region of the patient's ankle.

### DETAILED DESCRIPTION OF THE DRAWINGS:

The present invention provides a therapeutic sleeve for applying pressure to a body part. As such, the present therapeutic sleeve can be used under a compression sleeve, under bandages, or with any other compression system.

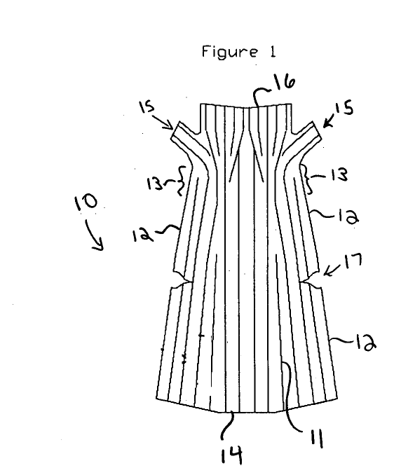

FIG. 1 is a plan view of the outer surface of an arm version of the present therapeutic sleeve showing the shape of the material before it is assembled into a tubular sleeve, showing stitches passing therealong. Specifically, sleeve 10 is made from a flat piece of semi-elastic material, such as foam. Such foam may be covered by a shell, including, but not limited to, a cotton-Lycra shell. In addition, or alternatively, a fabric laminate may be placed onto the foam. In various embodiments, the shell may be glued directly onto the foam, or the foam may simply received within the shell, which is then sewn shut over the foam.

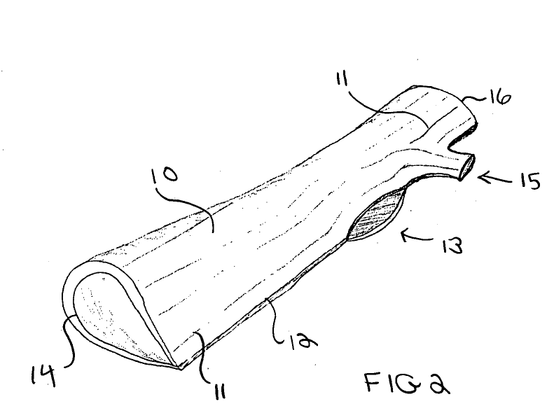

FIG. 2 shows a perspective view of the assembled arm version of the present therapeutic sleeve of FIG. 1. Specifically, to form the sleeve of FIG. 2, the flat piece of material shown in FIG. 1 is simply sewn (or otherwise fastened together) together along its side edges 12. Proximal end 14 and distal end 16 are left open, thus forming a tubular shaped device that is dimensioned to wrap around a body part (received therein).

As can also be seen in FIG. 2, an access opening 13 may be provided near the patient's wrist. Access opening 13 may simply be formed by not sewing together edges 12 in this region. Access opening 13 is used to insert additional padding so as to provide additional support and /or compression at locations adjacent to a patient's wrist.

A thumb opening 15 can be provided by not sewing together edges 12 in this region as shown. In optional embodiments, the thumb portion may instead be enclosed by sewing together edges 12 in this region. In further optional embodiments, the entire extending thumb covering portion may be removed, by simply providing a hole through which the patient's thumb extends.

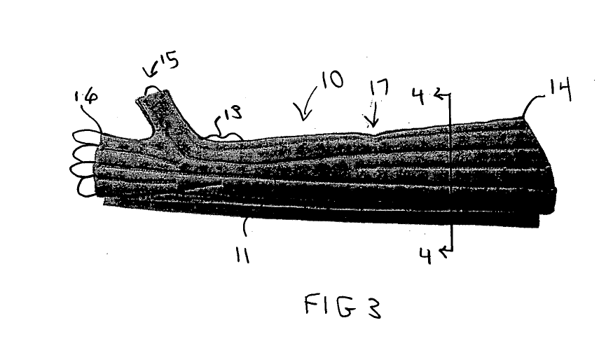

FIG. 3 illustrates the arm version of the present therapeutic sleeve showing its positioning on a patient's arm. As can be seen in FIGS. 1 and 3, a cut-out region 17 may be provided at the patient's elbow. Cut-out region 17 can accommodate any bunching of material that could otherwise occur at the interior of the elbow when the arm is bent. Removal of material circumscribed by cut-out region 17 thus reduces the potential for bunching of the therapeutic sleeve at the interior of the patient's elbow region.

Seams 11 are seen in each of FIGS. 1 to 4. As illustrated, therapeutic sleeve 10 has a plurality of seams 11 extending therealong. These seams 11 may extend in the longitudinal direction (as shown). However, the present invention is not so limited, as seams may extend in different directions. Moreover, it is to be understood that the present invention is not limited to any particular number or pattern of seams.

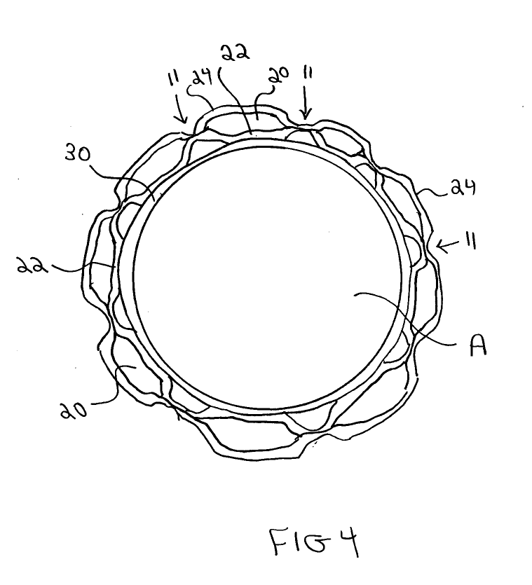

FIG. 4 is a sectional elevation view (taken along line 4-4 in FIG. 3) of the therapeutic sleeve 10 received over a patient's arm A. As can be seen, therapeutic sleeve 10 may comprise a foam portion 20, an inner shell 22 and an outer shell 24. As can be seen, seams 11 define regions of narrowed thickness through therapeutic sleeve 10. As illustrated, seams 11 may simply be formed by stitches passing through the tubular semi-elastic body along the length of the tubular semi-elastic body. Seams 11 have the advantage of making sleeve 10 conform properly to the shape of the patient's limb. This is true even as the patient's limb decreases in diameter over time. An optional inner sleeve 30 may also be provided if desired. In use, sleeve 30 is first pulled onto the patient's arm, and then therapeutic sleeve 10 is received thereover. Optional washable inner sleeve 30 protects sleeve 10 from getting dirty, and may also be made of a material to reduce friction when therapeutic sleeve 10 is pulled thereover.

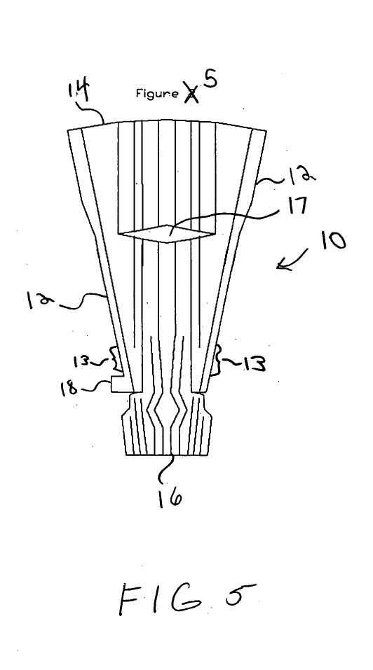

FIG. 5 shows a leg version of therapeutic sleeve 10 before it is assembled into a tubular sleeve. The leg embodiment of therapeutic sleeve 10 shown in FIG. 5 operates similar to the arm embodiment of therapeutic sleeve 10 shown in FIG. 2, as follows.

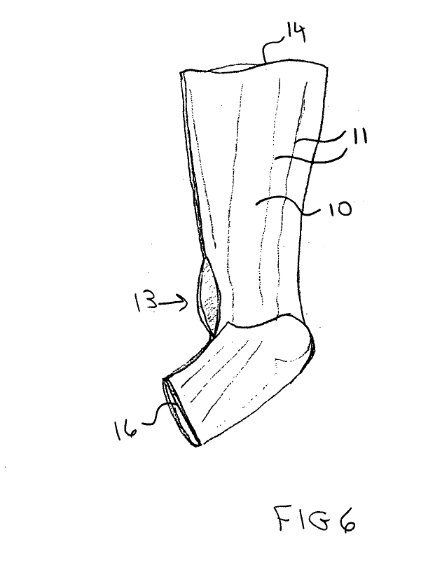

To form a sleeve like that shown in FIG. 6, a flat piece of material similar to that shown in FIG. 5 is simply sewn (or otherwise fastened together) together along its side edges 12. Proximal end 14 and distal end 16 are left open, thus forming a tubular shaped sleeve. (Note: FIG. 5 shows a full leg embodiment of the sleeve, whereas FIG. 6 shows an embodiment that only extends up to below the patient's knee.)

As can also be seen in FIG. 6, an access opening 13 may be provided near the patient's ankle. Access opening 13 may simply be formed by not sewing together edges 12 in this region. Access opening 13 is used to insert additional padding so as to provide additional support and /or compression at locations adjacent to a patient's ankle.

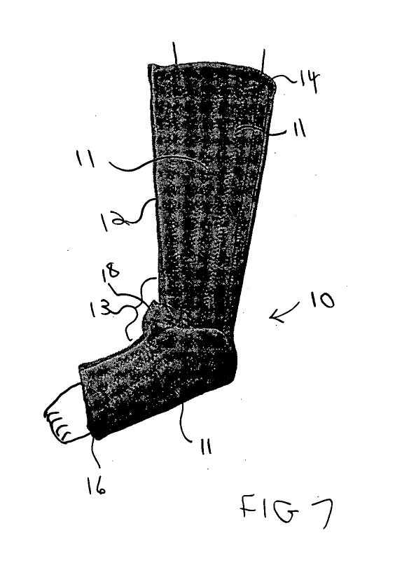

FIG. 7 illustrates the leg version of the present therapeutic sleeve showing its positioning on a patient's leg. As can be seen in FIG. 5, a cut-out region 17 may be provided at the back of the patient's knee. Cut-out region 17 can accommodate any bunching of material on the interior (back) of the knee that could occur when the knee is bent. Removal of material circumscribed by cut-out region 17 reduces bunching of the therapeutic sleeve by allowing the device to contour to the back of the knee region. An optional tongue 18 may be provided that can be inserted into an opening between the foot and the leg portions of the sleeve, as desired. Tongue 18 may be used to cover any gap or opening in this region, as needed. In optional embodiments, optional tongue 18 may be cut away from sleeve 10.

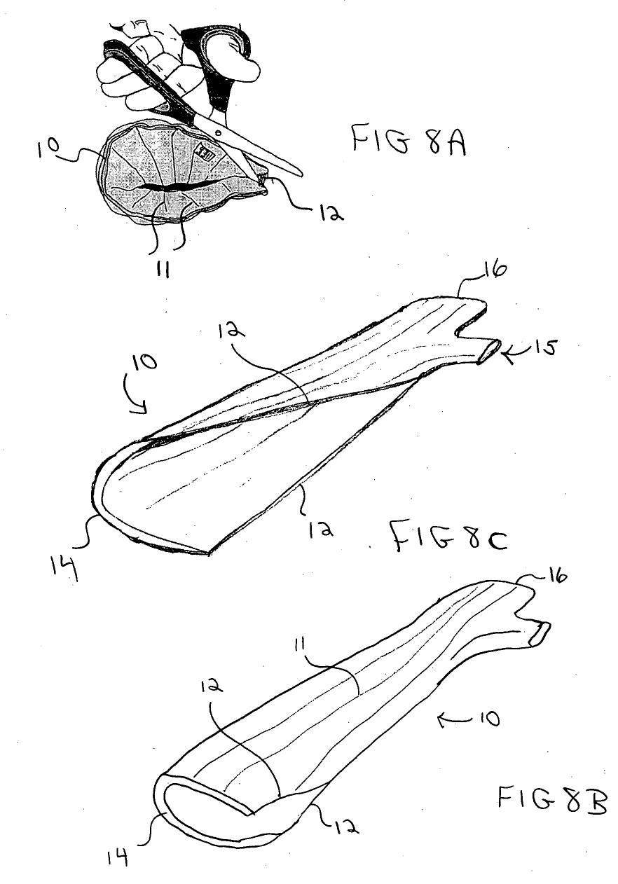

FIGS. 8A and 8B illustrate a method of sizing therapeutic sleeve 10 to fit the arm length of a particular patient. Specifically, sides 12 are sewn together (to form tubular therapeutic sleeve 10) by a reinforced seam. Thus, an operator need only cut open the reinforced seam connecting sides 12 to form an an openable slit extending longitudinally away from proximal end 14 of therapeutic sleeve 10. By opening this slit, as shown in FIG. 8A, the user can prevent bunching of sleeve 10 in their underarm region. Moreover, the slit can be cut open to different lengths to eliminate bunching near the patient's armpit such that sleeve 10 can be comfortably sized to patients of different arm lengths. For example, FIG. 8C illustrates an embodiment of the invention in which the sleeve is opened right down to the near the patient's hand. Such an embodiment may be useful should a patient desire to wrap part of sleeve 10 over upon itself, for example, after their limb has already decreased in diameter as a result of successful therapy.

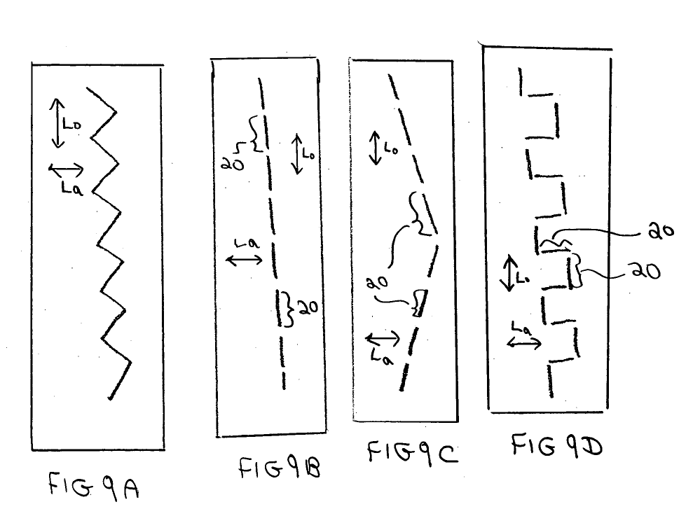

Seams 11 are formed by stitches passing through the foam material of therapeutic sleeve 10. Turning next to FIGS. 9A to 9D, the particular stitching pattern used in the present invention advantageously allows the sleeve to maintain its semi-elastic properties in perpendicular longitudinal and lateral directions. For example, FIG. 9A illustrates a close-up plan view of a zig-zag stitching pattern. This zig-zag stitching pattern permits sleeve 10 to flex both in the longitudinal (Lo) and lateral (La) directions.

In various embodiments of the invention, seams 11 are formed by stitches disposed in stitched sections 20 that are separated end to end from one another. FIGS. 9B, 9C and 9D show various embodiments of such stitching patterns. Specifically, such stitched sections 20 may be disposed in straight lines (FIG. 9B), semi-straight lines (FIG. 9C) or even at right angles to one another (FIG. 9D). Each of these stitching patterns permit sleeve 10 to flex in both the longitudinal (Lo) and lateral (La) directions.

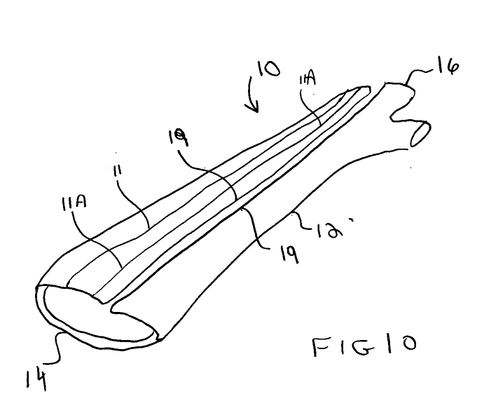

FIG. 10 shows an embodiment similar to FIG. 8B, but with a reinforced seam 19 being cut fully open from proximal end 14 to distal end 16 of sleeve 10. Thus, the entire sleeve 10 can be opened to wrap around the patient's limb and overlap upon itself. This may be useful in treating patients as their limbs decrease in diameter over time. In addition, sleeve 10 may be made such that longitudinal sections can be removed. For example, an operator could open the entire length of sleeve 10 by cutting along seam 19. Optionally, the user could cut out a section of material adjacent to seam 19. Such optionally cut out section of material may span along the entire length of the sleeve, but need not do so. For example, the user may cut fully along a seam 11A to remove a section of sleeve 10. As shown, seam 11A may be parallel to seam 19. It is to be understood, however, that the present invention is not so limited. Other removable sections are also contemplated within the scope of the present invention.

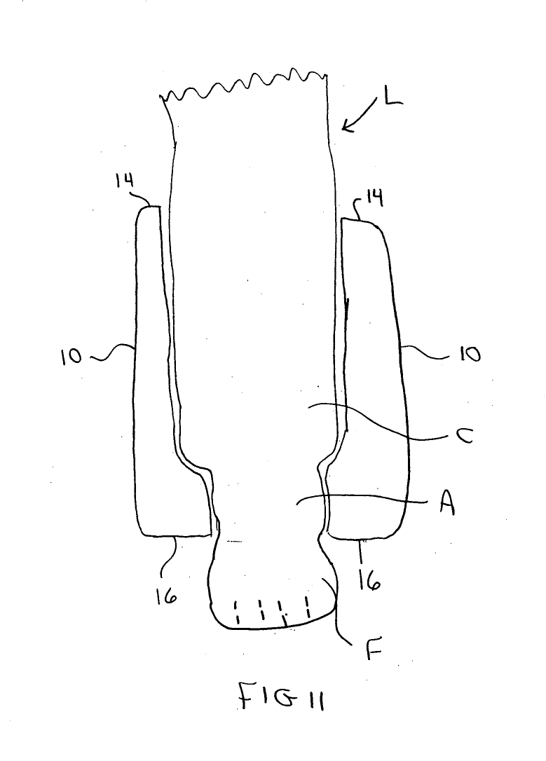

In optional embodiments, the thickness of therapeutic sleeve 10 may vary along its length. This may be achieved by providing a sleeve 10 that is made of separate material layers, some of which extend different lengths along the sleeve. For example, FIG. 11 shows an embodiment of the invention in which sleeve 10 has a different thickness along its length. Specifically, sleeve 10 may be thicker towards distal end 16 (as illustrated). This may be particularly advantageous in the case of a patient having a leg L with a large diameter calf C and a small diameter ankle A. Foot F is also shown. In this embodiment of the invention, additional support is provided to the patient's ankle A region by the increased thickness of sleeve 10 in this area. In optional embodiments, sleeve 10 may have a greater thickness near the patient's knee. Other possibilities of non-uniform sleeve thickness are also contemplated within the scope of the present invention.

As can be seen in FIGS. 1 and 5, therapeutic sleeve 10 may be made wider at its proximal edge 14 than its distal edge 16. This allows therapeutic sleeve 10 to accommodate the larger circumference of proximal limb segments. Also, by tapering the width of therapeutic sleeve 10, or otherwise varying its circumference, therapeutic sleeve 10 can be made to best conform to the shape of the body part.

In further optional embodiments, the seams of the present invention are formed from elastic threads. Such elastic threads may be used in embodiments of the present invention in which an access opening is, or is not, provided in the side of the sleeve. In its various embodiments, the use of elastic threads in seams 11 assists therapeutic sleeve 10 in conforming to the shape of the patient's body, and in providing strength and flexibility in lateral and longitudinal directions.

### Therapeutic Use

The present invention may be used to treat a variety of medical disorders which require compression therapy. Such disorders include, but are not limited to, lymphedema, phlebitis, varicose veins, post-burn treatment, post-fracture and injury (including sports injury such as a pulled muscle) edema, stasis ulcers, obesity and circulatory disorders. Moreover, the present invention may be used on both humans or animals, as desired.

After the sleeve placed on the body part, a compressive force or support can then be applied to the body part for a sufficient amount of time to mitigate swelling in the limb. Because human skin is elastic in nature, when such systems as the lymphatic or venous return systems fail to function properly, the limb or body part accumulates fluid and stretches to accommodate edema. Under normal operation, those systems would allow that fluid to circulate and not collect in those limbs or body parts and the skin would normally accommodate only the subtle changes by expanding or contracting. Use of various compression devices applied over the present invention maintains the lymph volume, not allowing the skin to stretch and accumulate fluid. The fluid must then flow through the system from the force of the applied compression device.

The present invention has been shown in preferred forms and by way of example, and many variations and modifications can be made therein within the spirit of the invention. The present invention, therefore, is not intended to be limited to any specified form or embodiment, except in so far as such limitations are expressly set forth in the claims.

What is claimed:

1. A therapeutic sleeve for applying pressure to a body part, comprising:
a tubular semi-elastic body;
at least one seam extending along the tubular semi-elastic body, the at least one seam defining a region of narrowed thickness of the tubular semi-elastic body; and
an access opening through a side of the tubular semi-elastic body.

2. The therapeutic sleeve of claim 1, wherein the at least one seam is formed by stitches passing through the tubular semi-elastic body.

3. The therapeutic sleeve of claim 1, wherein the at least one seam extends longitudinally along the length of the tubular semi-elastic body.

4. The therapeutic sleeve of claim 1, wherein the at least one seam comprises a plurality of seams defining regions of narrowed thickness extending along the length of the tubular semi-elastic body.

5. The therapeutic sleeve of claim 1, wherein the access opening is dimensioned to be disposed near a body joint when the therapeutic sleeve is received over the body part.

6. The therapeutic sleeve of claim 5, wherein the body joint is a wrist and the body part is an arm.

7. The therapeutic sleeve of claim 5, wherein the body joint is an ankle and the body part is a leg.

8. The therapeutic sleeve of claim 1, wherein the tubular semi-elastic body is dimensioned to wrap around the body part.

9. The therapeutic sleeve of claim 1, further comprising:
padding dimensioned to be inserted through the access opening.

10. The therapeutic sleeve of claim 1, wherein the tubular semi-elastic body is made of foam.

11. The therapeutic sleeve of claim 10, wherein the tubular semi-elastic body comprises a cotton-Lycra shell:

12. The therapeutic sleeve of claim 10, further comprising:
a fabric laminate on the foam.

13. The therapeutic sleeve of claim 1, further comprising:
an openable slit extending longitudinally from an end of the tubular semi-elastic body.

14. The therapeutic sleeve of claim 13, wherein the openable slit extends from the proximal end to the distal end of the tubular semi-elastic body.

15. The therapeutic sleeve of claim 13, wherein the openable slit comprises:
a reinforced seam configured to be cut open.

16. The therapeutic sleeve of claim 1, further comprising:
a detachable thumb portion receivable onto the tubular semi-elastic body.

17. The therapeutic sleeve of claim 1, further comprising:
a detachable tongue portion receivable onto the tubular semi-elastic body.

18. The therapeutic sleeve of claim 1, wherein the thickness of the tubular semi-elastic body varies along its length.

19. The therapeutic sleeve of claim 18, wherein the tubular semi-elastic body is formed from a plurality of separate material layers.

20. A therapeutic sleeve for applying pressure to a body part, comprising:
a tubular semi-elastic body;
at least one seam defining a region of narrowed thickness extending along the tubular semi-elastic body, the seam being formed by stitches passing through the tubular semi-elastic body, wherein the stitches are disposed in stitched sections that are separated end to end from one another.

21. The therapeutic sleeve of claim 20, wherein the stitched sections are separated end to end from one another longitudinally along the length of the tubular semi-elastic body.

22. The therapeutic sleeve of claim 20, wherein the stitched sections are disposed in a straight line.

23. The therapeutic sleeve of claim 20, wherein the stitched sections are disposed in a zig-zag pattern.

24. The therapeutic sleeve of claim 20, wherein successive stitched sections are disposed at right angles to one another.

25. The therapeutic sleeve of claim 20, wherein the tubular semi-elastic body is dimensioned to wrap around a body part.

26. The therapeutic sleeve of claim 20, wherein the at least one seam comprises a plurality of seams defining regions of narrowed thickness extending along the length of the tubular semi-elastic body.

27. The therapeutic sleeve of claim 20, wherein the tubular semi-elastic body is made of foam.

28. The therapeutic sleeve of claim 20, wherein the tubular semi-elastic body comprises a cotton-Lycra shell:

29. The therapeutic sleeve of claim 27, further comprising:
a fabric laminate on the foam.

30. The therapeutic sleeve of claim 20, further comprising:
an openable slit extending longitudinally from an end of the tubular semi-elastic body.

31. The therapeutic sleeve of claim 30, wherein the openable slit comprises:
a reinforced seam configured to be cut open.

32. The therapeutic sleeve of claim 20, further comprising:
a detachable thumb portion receivable onto the tubular semi-elastic body.

33. The therapeutic sleeve of claim 20, further comprising:
a detachable tongue portion receivable onto the tubular semi-elastic body.

34. The therapeutic sleeve of claim 20, wherein the thickness of the tubular semi-elastic body varies along its length.

35. The therapeutic sleeve of claim 20, wherein the tubular semi-elastic body is formed from a plurality of separate material layers.

36. The therapeutic sleeve of claim 1, wherein the at least one seam is formed by stitches passing through the tubular semi-elastic body, and wherein the stitches are disposed in stitched sections that are separated end to end from one another.

37. The therapeutic sleeve of claim 20, further comprising:
an access opening through a side of the tubular semi-elastic body.

38. A method of using a therapeutic sleeve for applying pressure to a body part, comprising:
receiving a body part within a tubular semi-elastic body having at least one seam extending therealong, the at least one seam defining a region of narrowed thickness of the tubular semi-elastic body; and
inserting padding through an access in a side of the tubular semi-elastic body.

39. The method of claim 38, wherein the padding is inserted near a body joint.

40. The method of claim 39, wherein the body joint is an ankle.

41. The method of claim 39, wherein the body joint is a wrist.

42. The method of claim 39, wherein the body joint is a knee.

43. The method of claim 39, wherein the body joint is an elbow.

44. A method of sizing a therapeutic sleeve to a body part, comprising:
receiving a body part within a tubular semi-elastic body having at least one seam extending therealong, the at least one seam defining a region of narrowed thickness of the tubular semi-elastic body; and
opening an end portion of the at least one seam.

45. The method of claim 44, wherein opening an end portion of the at least one seam comprises:
cutting open an end portion of a reinforced seam.

46. The therapeutic sleeve of claim 1, further comprising:
a cut-out region to facilitate bending of the body part.

47. The therapeutic sleeve of claim 46, wherein the body part is a leg, and wherein the cut-out region is disposed behind a patient's knee.

48. The therapeutic sleeve of claim 46, wherein the body part is an arm, and wherein the cut-out region is disposed at an interior side of a patient's elbow.

49. The therapeutic sleeve of claim 14, further comprising:
a removable portion of the sleeve adjacent to the openable slit.

50. The therapeutic sleeve of claim 20, further comprising:
a cut-out region to facilitate bending of the body part.

51. The therapeutic sleeve of claim 50, wherein the body part is a leg, and wherein the cut-out region is disposed behind a patient's knee.

52. The therapeutic sleeve of claim 50, wherein the body part is an arm, and wherein the cut-out region is disposed at an interior side of a patient's elbow.

53. The therapeutic sleeve of 30, further comprising:
a removable portion of the sleeve adjacent to the openable slit.

54. The method of claim 44, further comprising:
removing a portion of the therapeutic sleeve to fit the sleeve to the body part.

55. The method of claim 54, wherein the removed portion of the therapeutic sleeve extends along the entire length of the therapeutic sleeve.

56. A therapeutic sleeve for applying pressure to a body part, comprising:
a tubular semi-elastic body; and
at least one seam extending along the tubular semi-elastic body, the at least one seam defining a region of narrowed thickness of the tubular semi-elastic body, wherein the at least one seam is formed from an elastic thread.
